(19) United States
(12) Patent Application Publication
(10) Pub. No.: US 2005/0053991 A1
(43) Pub. Date: Mar. 10, 2005

(54) Mutant cells with enhanced resistance to desiccation

(76) Inventors: Alan Greener, San Diego, CA (US); James Jolly, St. Charles, IL (US); Latha Sundar, San Diego, CA (US)

Correspondence Address: PALMER & DODGE, LLP;KATHLEEN M. WILLIAMS / STR, 111 HUNTINGTON AVENUE, BOSTON, MA, 02199, US

(21) Appl. No.: 10/942,279

(22) Filed: Sep. 15, 2004

Related U.S. Application Data
(63) Continuation of application No. 10/016,921, filed on Dec. 14, 2001, now abandoned.
(60) Provisional application No. 60/256,093, filed on Dec. 15, 2000.

(51) Int. Cl. C12Q 1/02

(52) U.S. Cl. 435/6; 435/29

(57) ABSTRACT

The invention provides a method of isolating desiccation resistant cells which retain good viability over long periods of time at room temperature for a variety of applications. In one embodiment, competent cells for DNA transformation are made from selected bacterial cells which are storage stable at room temperature.

### RELATED APPLICATIONS

This application is a continuation of U.S. application Ser. No. 10/016,921, filed Dec. 14, 2001, which claims priority under 35 U.S.C. 119(e) to U.S. Provisional Application Ser. No. 60/256,093 filed Dec. 15, 2000, the entirety of which are incorporated herein by reference.

### FIELD OF THE INVENTION

The invention relates to storage-stable cells which retain viability over long periods of time and to methods of generating such cells. Specifically, it relates to cells that are resistant to denaturation and other destructive reactions caused by desiccation and to methods of generating such cells. The invention also relates to transformation-competent cells made from these storage-stable cells and to methods of generating such cells.

### BACKGROUND OF THE INVENTION

Microorganisms and other types of cells have a broad range of commercial applications. Such applications include agriculture (e.g., as plant pathogen inhibitors), aquaculture (e.g., in the biodegradation of uneaten feed and excreta), the food industry (e.g., in food fermentation), environmental applications (e.g., such as clean-up of oil spills and liquid-based pollution), the biomedical/pharmaceutical industries (e.g., as vaccines), and laboratory research (e.g., as sources of competent cells).

In order to provide convenient sources of cells for these applications, it is desirable to provide long-term storage conditions which maintain the viability and biological functions of these cells. Typically, cells are stored in an aqueous suspension at a temperature of −80° C. or below for long periods of time. These low temperatures minimize detrimental reactions such as oxidation and attack by free radicals (Mouradian, et al., Cryobiology 22: 119-127, 1985). However, there are problems associated with low temperature storage, including the high cost of maintaining low temperature storage units and limited accessibility to these units. Further, it is difficult to transport cells under suitable low temperature conditions.

Thus, it is advantageous to be able to store cells at higher temperatures, such as room temperature. However, when biological materials are stored at high temperatures in an aqueous suspension state for an extended time, they are susceptible to the detrimental reactions caused by water and oxygen. These reactions significantly reduce cell viability and function, and therefore, the subsequent usefulness of the cells. By removing detrimental agents such as water and oxygen, desiccation can minimize cell damage, enabling cells to be more stable at higher temperatures. At the same time, storing cells in a dry state also reduces the costs associated with maintenance and transportation. However, most types of cells are sensitive to denaturation, osmotic shock, and other destructive reactions caused by desiccation which result in loss of viability (Souzu, et al., Biochim Biophys Acta 978: 112-118, 1989). For bacterial cells that are used as competent cells, a loss of transformation efficiency is also associated with the loss of viability.

Previous attempts to obtain biologically functional dehydrated cells have been described, most of which focus on drying cells at freezing temperature (“freeze-drying”). Patents by Goodrich, et al. (U.S. Pat. Nos. 5,648,206 and 5,340,592) disclose a process of freeze-drying cells, preferably erythrocytes, in the presence of monosaccharides and other biocompatible polymers. The monosaccharides and biocompatible polymers provide an isotonic aqueous solution and allow the cells to maintain their structural and functional integrity during the drying process. Japanese Patent No. 6,126,508,5 A2 discloses a method of freeze-drying bacteria in the presence of stabilizing compounds such as phenylalanine, histidine, citric acid, succinic acid, tartaric acid, and alkali carbonate. Cells dried by this method remain viable for up to 30 days at 35° C.

In general, methods of obtaining storage-stable cells have focused on modifying the parameters of the drying and reconstitution process. For example, U.S. Pat. No. 5,733,774 describes a method of producing dormant bacterial cells that can survive long-term storage at temperatures of between 5° C. to 30° C. In this method, both water and oxygen are removed from bacterial cultures drying at temperatures between 40° C. to 140° C. in the presence of oxygen-depleting agents. The bacterial cells are reported to remain stable at temperatures between 5° C. and 30° C. for at least a year. The efficacy of the stored bacterial cell as a biocontrol agent for inhibiting a plant pathogen, Penicillium expansium, is also preserved.

WO 98/35018 discloses a method of lyophilizing transformation-competent cells to generate cells which are stable at −20° C. for up to a year. In this method, cells which have been previously frozen at temperatures from −20° C. to −180° C. are dried in the presence of a cryoprotectant. During drying, the cells are exposed to a series of temperature steps from −45° C. to 11° C. at a rate of about 0.1° C. to 1.0° C./hour. The publication reports that the cells are stable at a range of temperatures, including room temperature and that the cells retain transformation efficiencies of 1×10⁵ to 1×10⁹ transformants/1 g of DNA.

Attempts to genetically modify cells which are storage-stable at higher temperatures have also been described. For example, U.S. Pat. No. 5,891,692 describes a method of modifying bacterial cells by transforming the cells with exogenous E. coli genes involved in fatty acid synthesis. The modified cells have increased unsaturated fatty acid content allowing them to be stored at −20° C. to 4° C. in aqueous suspensions without significantly decreasing viability and transformation efficiencies.

### SUMMARY OF THE INVENTION

The present invention relates to a method of isolating storage-stable mutant cells that are resistant to desiccation by exposing cells to one or more desiccation processes and selecting cells which have enhanced viability after these processes compared to non-mutant cells. The method permits stable storage of the mutant cells in a dried form at temperatures higher than −80° C., e.g., at least 4° C., and preferably at room temperature (15° C.-40° C.). Competent cells made from bacterial mutants according to the invention can be stored at higher temperatures without appreciably losing their viability and transformation efficiencies. In one embodiment, greater than 10% of the cells remain viable after drying. In another embodiment, the cells retain transformation efficiencies of at least 10⁵ transformants/μg DNA.

The method of the invention comprises growing the cells in a cell-growth medium at an appropriate temperature (e.g., 10° C. to 40° C.), collecting and resuspending the cells, drying, and selecting cells that have increased resistance to loss of viability after desiccation, and increased resistance to desiccation stresses. In one embodiment, cells are mutagenized prior to drying.

In one embodiment, at least 10%, at least 20%, or at least 30% of the mutant cells are able to survive desiccation. In another embodiment, at least 40%, at least 50%, at least 60%, at least 70%, at least 80%, or at least 90% of the mutant cells survive desiccation. In a further embodiment, 100% of the cells survive desiccation. In another embodiment, cells are selected which show enhanced transformation efficiencies compared to non-mutant cells when these cells are rendered competent (e.g., 10⁶-10¹² transformants/μg DNA).

The invention further provides a method of identifying mutant cells with enhanced resistance to desiccation. In one embodiment, the method comprises mutagenizing a population of cells (e.g., using ultraviolet light or a chemical mutagen), exposing the population to drying, selecting cells which survive drying, and subjecting the cells which survive to at least one additional round of drying to identify clones of cells which have enhanced survival (e.g., greater than 10%, and preferably at least 30% viability). In one embodiment of the invention, cells are dried at a non-freezing temperature for 6-48 hours, and preferably 8-16 hours. In one embodiment, the cells are dried at temperatures from 4° C. to 30° C., and most preferably at temperatures within the range of 20° C.-30° C. In a preferred embodiment, cells are dried at a uniform temperature, without temperature steps. In a further embodiment, cells are subjected to at least 2, at least 4, or at least 6, cycles of drying and selection, to identify cells with enhanced viability.

In one embodiment, cells are exposed to drying in the presence of a glass-forming matrix.

In one embodiment according to the invention, the glass-forming matrix material comprises a carbohydrate or derivative thereof. In another embodiment, the concentration of the carbohydrate or derivative thereof is at least 20% (weight/volume). In one embodiment, the glass forming matrix comprises a non-reducing sugar, such as a disaccharide or a trisaccharide. In a further embodiment of the invention, the sugar is selected from the group consisting of trehalose, sucrose, maltitol, melezitose, raffinose, alcohol derivatives thereof and combinations thereof.

In one embodiment, the storage-stable mutant cells can be stored at temperatures above −80° C. and when rendered competent, can maintain transformation efficiencies of at least 10⁵ transformants/μg DNA for at least one month. In another embodiment, the cells can be stored at temperatures of −20° C. or above and when rendered competent, can maintain transformation efficiencies of at least 10⁵ transformants/μg DNA for at least one month. In still another embodiment, the cells can be stored at temperatures of 0° C., at 4° C., at 15° C., at 20° C., or at room temperature, or above, and when rendered competent can maintain transformation efficiencies of at least 10⁵ transformants/μg DNA for at least one month. In a further embodiment of the invention, mutant cells are selected for which, in addition to having enhanced resistance to desiccation, have enhanced survivability after exposure to transformation buffers (e.g., CaCl₂ or electroporation buffers).

In one embodiment, cells are dried under vacuum. In a further embodiment, cells are dried under vacuum for 2-24 hours at room temperature (e.g., from 15-30° C.). Still more preferably, cells are dried at 30° C. for 6-48 hours. In a further embodiment according to the invention, storage stable mutant cells are provided which, when transformed, maintain a transformation efficiency of 1×10⁵-1×10¹² transformants/μg DNA for greater than a month at room temperature.

Preferably, the storage-stable mutant cells according to the invention are microorganisms. More preferably, the cells are bacterial cells. Yet more preferably, the cells are gram negative bacterial cells. In one embodiment, the cells are E. coli cells. In still another embodiment, the cells are made competent for transformation of exogenous DNA.

### DESCRIPTION

The mutant cells according to the invention show enhanced resistance to desiccation and are also more storage stable at room temperature. In one embodiment, desiccation-resistant mutant cells are provided which can be stored at temperatures higher than −80° C. for long periods of time (e.g., at least for one month) without appreciable loss of viability. The cells can therefore be maintained and transported at temperatures higher than 0° C. without the use of ice or other frozen packaging materials. In a further embodiment, the mutant cells can also be used to generate room temperature stable competent cells for transformation with exogenous nucleic acids.

### Definitions

In order to more clearly and concisely describe and point out the subject matter of the claimed invention, the following definitions are provided for specific terms that are used in the following written description and the appended claims.

As used herein, “mutation” refers to any alteration of the genetic constitution of a cell by changing the structure of the cell's hereditary material, deoxyribonucleic acid (DNA).

As defined herein, a “mutagen” is any agent or condition that can induce a mutation at frequencies greater than the spontaneous mutation rate for the organism.

As defined herein, “mutagenesis” refers to the process of generating a mutation.

As defined herein, “mutants” or “mutant cells” are cells which comprise one or more mutations.

As defined herein, “biological agent” refers to a agent containing DNA or RNA and having biological functions such as infecting a cell, integrating into hereditary material of a cell, and replicating inside a cell.

As used herein, “selection” refers to a process to enriching for cells with desired heritable characteristics from a cell population, (e.g., such as resistance to desiccation, enhanced transformation efficiencies, and/or enhanced viability upon exposure to a transformation buffer) by identifying cells in a population with a desired characteristic.

As used herein, “clonal progeny” refers to genetically identical cells which result from the cell division of a isolated desiccation-resistant mutant cell.

As used herein, “without appreciably losing viability and transformation efficiency” refers to a greater than 10% viability after desiccation and rehydration and a transformation efficiency of greater than 10⁵/μg DNA, when cells are rendered competent. Transformation efficiencies will, of course, vary depending upon the size of the transforming plasmid. Thus, transformation efficiency of cells according to the invention may be different for different size plasmids. A plasmid of 1.0 kb is expected to have a transformation efficiency of 10¹² transformants/μg DNA; a plasmid of 2.6 kb (e.g., pUC18) is expected to have a transformation efficiency of 3.3×10¹¹ transformants/1 g DNA; a plasmid of 5.0 kb is expected to have a transformation efficiency of 1.6×10¹¹ transformants/μg DNA.

As used herein, “storage-stable” refers to cells that are able to withstand storage conditions above −80° C. for extended periods of time (e.g., at least one month) without appreciably losing their viability (e.g., greater than 10% of the cells survive). For competent cells, it also refers to such cells that are able to retain transformation efficiencies of at least 10⁵ transformants/μg DNA after extended periods of storage (e.g., for at least one month).

As used herein, “desiccation” refers to a process which removes intercellular or intracellular water. The water removed can be free water between or inside the cells; or water bound to macromolecules (e.g., such as proteins, nucleic acids, cell membrane molecules or other substances inside the cells) and/or water preserved in the pockets of lipids and membranes. The terms “drying” and “desiccating” are used synonymously.

As used herein, a “desiccation-resistant cell” is a cell which exhibits higher viability after drying than a non-desiccation-resistant cell.

As used herein, “competent cell” refers to a cell that has the ability to take up and amplify an exogenous nucleic acid.

As used herein, the term “positive genomic clone” refers to a genomic DNA clone that transforms and enables a cell which receives the DNA to become resistant to dehydration.

Cells

A variety of cells can be used to isolate desiccation-resistant mutants, and include, but are not limited to, microorganisms, such as fungi and bacteria. Bacteria encompassed within the scope of the invention include, but are not limited to, gram negative and gram positive bacterial cells which can be made competent for transformation by exogenous DNA (either using chemical agents or by electroporation), such as Eschericia sp. (e.g., E. coli), Klebsiella sp., Salmonella sp., Bacillus sp., Streptomyces sp., Streptaococcus sp., Shigella sp., Staphylococcus sp., and Pseudomonas sp. Suitable E. coli strains include, but are not limited to, BB4, C600, DH5, DH5a, DH5a-E, DH5aMCR, DH5a5′IQ, DH5a5′, DH10, DH10B, DH10b/p3, DH10BAC, HB101, RR1, JV30, DH11S, DM1, LE392, SCSI, SCS110, Stab2, DH12S, MC1061, NM514, NM522, NM554, P2392, SURE®, SURE 2, STBL2™ Competent Cells or ELECTROMAX™ STBL4TM cells, XL1-Blue, XL1-Blue MRF, XL1-BlueMR, XL2-Blue, XL10-GOLD, JM101, JM109, JM110/SCS110, NM522, TOPP strains, ABLE®, XLI-Red, BL21, TK B11, XL10-Gold® Cells, Restriction-Minus Competent Cells™, TK Cells, ABLE® strain, XlmutS strains, SCS110, AGI, ElectroTen-Blue™ strains, TG1, SOLR™, XLOLR strain, Y1088, Y1089r, Y1090r-strains, WM100, and derivatives thereof. Information relating to the genotypes of these strains are known in the art and can be found, for example, at www.strategene.com.

Mutagenesis

Mutagenesis of cells can occur spontaneously or can be induced by mutagens. In one embodiment, cells are mutagenized by exposure to a mutagen prior to desiccation and selection. Suitable mutagens encompassed within the scope of the invention include, but are not limited to, ultraviolet rays, alpha, beta, gamma, and X radiation, extreme changes in temperature, and chemical agents such as nitrous acid, nitrogen mustard, nitrosoguanidine, sodium bisulfite, hydrazine, formic acid, 5-bromouracil, 2-aminopurine, other chemical substitutes for portions of the nucleotide subunits of genes, acridine, proflavine, acriflavine, quinacrine, hydroxylamine and ethidium bromide and combinations thereof. Biological agents such as retroviruses, transposable elements, and plasmids can also induce mutations in a cell, as well as non-biological agents, such as synthetic oligonucleotides. In still a further embodiment, mutator strains of bacteria may be used which comprise higher rates of spontaneous mutagenesis, with or without exposure to a mutagen. Methods of mutagenizing bacterial cells and mutator strains are routine in the art and are described, for example, in U.S. Pat. No. 6,156,509, U.S. Pat. No. 6,103,470, U.S. Pat. No. 4,980,288, Greener, et al., Strategies in Molecular Biology, vol. 7: pp. 32-34, 1994, Snyder, et. al, American Society for Microbiology, Chap. 3: pp. 85-89,1997, and Shafikani, et al., Bio Techniques 23(2): 304-310, 1997, the entireties of which are incorporated herein by reference.

Genetically engineered cells that exhibit and enhanced spontaneous mutation rate can also be used to generate mutants according to the invention.

In one embodiment, desiccation-resistant cells are provided which comprise one or more mutations which result in a desiccation-resistant phenotype (e.g., clonal progeny of a mutant cell exhibits greater than 10% viability after desiccation and rehydration). Types of mutations comprise: point mutations (e.g., induced by ultraviolet light), additions and deletions (e.g., induced by transposons,), chromosomal duplication, chromosomal breakage and realignment, and insertion of exogenous DNA (e.g., induced by retroviruses, cloned transposable elements, plasmids, or synthetic oligonucleotides).

In one embodiment, mutant cells are provided which comprise point mutations. In this embodiment, a suitable mutagen to generate such cells is ultraviolet (UV) light. UV induces point mutations primarily by inducing covalent linkages between adjacent pyrimidines, blocking the replication of DNA. UV induced mutations which remain uncorrected or corrected by error-prone mechanisms create heritable changes in an organism's genome. Therefore, in one embodiment, mutations are generated by exposing the cells to ultraviolet light UV light in the range of from 254 nm to 320 nm (see, e.g., as described in U.S. Pat. No. 5,711,977, Lehmann, Gene 253: 1-12, 2000; McGregor, J. Investig. Dermatol. Symp. Proc. 4: 1-5, 1999; Rattista, Basic Life Sci. 52: 269-275, 1990, the entireties of which are incorporated herein by reference).

In one embodiment, bacterial cells are first grown in a medium which supports cell proliferation (“cell-growth medium”) prior to exposure to a mutagen. Cell-growth medium encompassed within the scope of the invention, includes, but is not limited to: Luria Broth; Psi broth (e.g., 5 grams bacto yeast extract, 20 grams Bacto tryptone, 5 grams of magnesium sulfate, per liter); SOB medium (e.g., 0.5% yeast extract, 2% tryptone, 10 mM NaCl, 2.5 mM KCl, 10 mM MgCl₂, 10 mM MgSO₄); SOC medium (e.g., 2% tryptone, 5% yeast extract, 2.5 mM KCl, 10 mM NaCl, 10 mM MgCl₂, 20 mM glucose); Terrific Broth (“TB”) (e.g., 12 grams of tryptone, 24 grams of yeast extract, 4 ml of glycerol 2.3 grams of KH₂PO₄, 12.5 grams of K₂HPO₄, per liter); TY medium (8 grams of tryptone, 5 grams of NaCl, 5 grams of yeast extract, per liter, adjusted to pH 7.2-7.4 with NaOH), and other media used to support the growth of cells, such as bacteria. It should be obvious to those of skill in the art that a variety of media can be used, and that such media are encompassed within the scope of the invention.

Incubation temperatures for growing cells can vary from 10° C. to 42° C., but preferably ranges from 20° C. to 40° C. In one embodiment according to the invention, bacterial cells are grown with shaking to promote aeration, at 100 to 500 revolutions per minute (rpms). In a preferred embodiment, bacterial cells are grown to early to mid log phase, or to early stationary phase, or to late stationary phase, as detected by visual inspection (e.g., for optimal turbidity) or by sampling aliquots of media and determining the optical density (OD) of the media (e.g., at 550 nm, using a spectrophotometer) to select cells having an OD between 0.1 to 2.0. Media can be reinoculated with cells which have reached mid to late stationary phase to reinitiate log phase growth.

For mutant isolation, cells at any phase of the growth curve can be used, but preferably cells are mutagenized at early to mid log phase or early stationary phase. In one embodiment, a suspension of cells at a suitable growth stage is placed in a culture container and exposed to either a chemical or physical mutagen (e.g., UV light).

In one embodiment, mutagenized cells are directly exposed to the selection process (e.g., exposure to desiccation conditions followed by selection of viable cells). However, in another embodiment, cells are incubated from 1 to 48 hours after dilution in cell-growth medium to expand populations of mutant cells. If chemical mutagens have been used, the cells are collected (e.g., by centrifugation, filtering, allowing cells to settle, by size exclusion chromatography, and the like) and washed in cell-growth medium prior to further incubation.

In one embodiment, cells are collected prior to drying and resuspended, with or without at least one wash in a suitable buffer or medium. In another embodiment, bacterial cells are washed at least one time and resuspended in a transformation buffer, to additionally select cells which are both resistant to the desiccation process and exposure to transformation buffers.

Suitable transformation buffers include, but are not limited to, buffers comprising chemical agents for rendering cells chemically competent, such as 50 mM CaCl₂, 10 mM Tris/HCL (Sambrook, et al., Molecular Cloning: a Laboratory Manual, 2nd Edition, eds. Sambrook, et al., Cold Spring Harbor Laboratory Press, 1989); TB buffer (e.g., 10 mM PIPES, 15 mM CaCl₂, 250 mM KCl) (Inoue, et al, Gene 96: 23-28, 1990); 2×TSS (LB broth with 10% PEG (MW3350-8000), 5% DMSO, and 20-50 mM Mg²⁺ (MgSO₄ or MgCl₂) at a final pH of 6.5) (Chung, et al., PNAS 86: 2172-2175, 1989); FSB buffer (e.g., 10 mM-potassium acetate, 100 mM-KCl, 44 mM-MnCl₂, 10 mM-CaCl₂, 3 mM-HACoCl₃, 10% redistilled glycerol) (Hanahan, D., In: DNA Cloning (D. M. Glover, ed) IRL Press, Washington, D.C., pp. 109-135); and CCMB80 buffer (10 mM potassium acetate pH 7.0, 80 mM CaCl₂, 20 mM MnCl₂, 10 mM MgCl₂, 10% glycerol, adjusted to pH 6.4 with 0.1N HCl) (Hanahan, et al., Methods in Enzymology 204: 63-113, 1991). (The entirety of these references are incorporated herein by reference.) Preferably, when cells are rendered chemically competent for the selection procedures, the cells are resuspended in transformation buffer which has been pre-cooled to 4° C.

Cells can also be made competent by exposure to electrical pulses which create temporary holes in the cells' plasma membranes (Potter, Anal. Biochem. 174: 361-73 (1988); U.S. Pat. No. 6,096,549, the entireties of which are incorporated herein by reference). Therefore, in one embodiment, cells are grown in culture media to mid or late log phase or to early stationary phase, collected and then resuspended and washed at least one time in an electroporation buffer (e.g., 10%-15% glycerol, 90% distilled water, v/v), prior to exposure to drying and selection.

Mutant Selection

Cells are subjected to selection under desiccation conditions with, or without, prior exposure to mutagens. Different cells have different tolerance to desiccation (defined as sensitivity to different degrees of water removal), therefore selection procedures are customized according to which particular cell type is being mutagenized (see, e.g., Janning, et al., J. Appli. Bacteriol. 77: 319-324, 1994, the entirety of which is incorporated herein by reference). For example, for cell types with low desiccation tolerance, a shorter exposure to desiccation conditions can be used (e.g., 4-8 hours), while for cell types with high desiccation tolerance, a longer exposure to desiccation conditions can be used (e.g., greater than 24 hours).

A variety of drying methods can be used. These methods include, but are not limited to, freeze-drying, air-drying, vacuum-drying, oven-drying, spray-drying, flash-drying, fluid bed-drying, and controlled atmosphere drying and are described, for example, in U.S. Pat. No. 5,728,574; U.S. Pat. No. 5,733,774; U.S. Pat. No. 5,200,399; U.S. Pat. No. 5,340,592; and U.S. Pat. No. 4,797,364, the entireties of which are incorporated by reference). In one embodiment, cells are dried at temperatures above freezing. In another embodiment, cells are dried at temperatures greater than or equal to 4° C. In still a further embodiment, cells are dried at room temperature (e.g., from 15-40° C.) under vacuum for 2-24 hours (e.g., 16 hours). In one embodiment, cells are dried under vacuum at non-atmospheric pressure, e.g., 1000-4000 mtorr.

In a preferred embodiment, cells are dried in the presence of a glass-forming matrix material. Suitable glass-forming matrix materials include carbohydrates, such as non-reducing sugars, which minimize oxidative damage to the cells. In one embodiment, the matrix material is a saccharide selected from the group consisting of trehalose, sucrose, melzitose, raffinose, alcohol derivatives thereof, and combinations thereof. In a preferred embodiment, the competent cells are contacted with a 20% carbohydrate solution, such as 20% trehalose, 20% sucrose, 20% melzitose, or 20% raffinose. In one embodiment, the cells are exposed to a solution which comprises 10% of two different carbohydrate solutions (e.g., 10% trehalose and 10% melzitose; 10% raffinose and 10% trehalose; 10% raffinose and 10% melzitose; 10% trehalose and 10% sucrose; 10% raffinose and 10% sucrose; or 10% melzitose and 10% sucrose).

In a preferred embodiment, a saccharide is used which does not crystallize upon drying and which comprises a Tg in the range of 10° C. to 80° C. In one embodiment, the glass-forming matrix material is a non-reducing carbohydrate selected from the group consisting of disaccharides, trisaccharides, oligosacharides and sugar alcohols thereof. Preferred saccharides include, but are not limited to, trehalose, raffinose, melezitose, sucrose, maltitol or combinations thereof. In one embodiment, a glass-forming saccharide is selected which hydrolyzes into a reducing sugars at a slow rate (e.g., such as trehalose). In another embodiment, a saccharide is selected which forms a hydrate when water is absorbed, thereby maintaining a high Tg (>15° C., and preferably greater than 40° C.) upon drying.

Other glass-forming matrix materials are known and are encompassed within the scope of the invention. These include, but are not limited to, dextran, polyethylene glyol, ficoll, and the like.

In one embodiment, the viability of cells obtained in initial rounds of desiccation and selection (e.g., the first two rounds) is 5% to 15%, providing a suitable number of cells for subsequent selection cycles and analyses. In a further embodiment of the invention, cells are exposed to at least two rounds of desiccation and selection, at least four rounds of desiccation and selection, or at least six rounds of desiccation and selection. In one embodiment, the viability of cells obtained after, at least the final round of desiccation and selection, is greater than 10%, greater than 20%, greater than 30%, greater than 40%, greater than 50%, greater than 60%, greater than 70%, greater than 80%, greater than 90%, or 100%.

Cells selected for enhanced resistance to desiccation (e.g., by identifying viable cells at each round of desiccation) are isolated by collecting the surviving cells and obtaining an expanded clonal population of the same cells. In one embodiment, cells are isolated by plating onto plates comprising cell-growth medium (e.g., LB agar plates) and selecting individual colonies representing a clonal population of cells. In another embodiment, cells are isolated by limiting dilution to obtain a clonal population of cells. Either or both of these methods may be used. Colonies or clones identified are either individually cultured in liquid medium and stored, or are pooled and rehydrated in appropriate buffer or medium (e.g., cell-growth medium or transformation medium) for further rounds of desiccation and selection.

In one embodiment, mutant cells are selected by expanding individual colonies/clones identified in a first round of dehydration, individually drying populations of cells which represent clones of cells in a second round of desiccation, and identifying clones which have at least 10% viability upon rehydration, at least 20% viability, at least 30% viability, at least 40%, at least 50%, at least 60%, at least 70%, at least 80%, at least 90% or 100% viability. Clones with enhanced resistance to desiccation in the second round of screening (e.g., greater than 10% viability) are then exposed to further rounds of selection for to identify new clones with the same or higher viability.

In another embodiment, cells are subjected to rounds of desiccation and rehydration without determining the viability of individual clones at each round of desiccation, e.g., simply selecting for cells which survive the process without determining their percent viability upon dehydration. In one embodiment, cells are subjected to at least two rounds of desiccation and selection. In a preferred embodiment, cells are subjected to six rounds of desiccation and selection. At the end of a final round of selection, cells which survive are cloned by plating or limiting dilution and expanded by culturing. The cloned cells are then dried and their resistance to desiccation is tested by measuring their viability after rehydration. In one embodiment, the viability of cloned cells is greater than 10% upon rehydration. In another embodiment, the viability of cloned cells is at least 20%, at least 30%, at least 40%, at least 50%, at least 60%, at least 70%, at least 80%, at least 90%, or 100%, after drying and rehydration. In a further embodiment, the heritability of the desiccation-resistance phenotype is confirmed by subjecting cloned cells to at least one more round of desiccation and measuring viability.

In another embodiment of the invention, in addition to selecting for enhanced viability upon rehydration, cells are selected for enhanced storage stability (e.g., ability to maintain viability of greater than 10% over a period of at least one month) at temperatures greater than −80° C. In one embodiment, cells are selected which have enhanced storage stability for at least one month at room temperature (e.g., 15° C. to 40° C.).

In a further embodiment of the invention, in addition to selecting for enhanced viability, cells are selected which exhibit enhanced transformation efficiencies upon exposure to exogenous nucleic acids in the presence of transformation buffer or under other transforming conditions (e.g., exposure to at least one electrical pulse). In one embodiment, cells are selected which comprises transformation efficiencies of greater than 10⁵ transformants/μg DNA, greater than 10⁶ transformants/μg DNA, greater than 10⁷ transformants/μg of DNA, greater than 10⁸ transformants/μg DNA, greater than 10⁹ transformants/μg DNA, greater than 10¹⁰ transformants/μg DNA, greater than 1011 transformants/μg DNA, or greater than 10¹² transformants/μg DNA.

The invention further provides unique mutant cell strains which exhibit desiccation resistance. In one embodiment, these strains comprise XL1-Blue MRF mutants XA2, XA47, XA49, XB40, and XB46. In another embodiment, these strains comprise Electro Ten Blue mutants BA4 and BA98. In still another embodiment, the strains comprise XL10-GOLD mutants AT4, AT7, AT8, AT9, AT37, AT40, AT54, AT66, AT67, BT71, BT72, and BT87.

Mutant Characterization

In one embodiment, individual mutants are characterized to identify the nature of the mutation which confers enhanced resistance to desiccation and/or enhanced transformation efficiency when the cells are rendered competent. In one embodiment, bacterial cells are first tested for the presence of genetic markers that are characteristic of the parental (e.g., non-mutagenized) cells, such as antibiotic or metabolic markers which allow the bacteria to grow on medium containing specific antibiotics and/or metabolites.

In one embodiment, genomic DNA is then isolated from individual bacterial isolates as described by Lin and Kuo, Focus 17: 66-70, 1995, the entirety of which is herein incorporated by reference. A genomic DNA library is constructed using methods known to those skilled in the art, such as described in Sambrook, et al., supra, the entirety of which is incorporated herein by reference and at http://www.protocol-online.net/molbio/DNA/dna_library.htm, after obtaining fragments of genomic DNA from the clonal progeny of a desiccation-resistant mutant cells. Bacterial cells comprising DNA fragments from the genomic library are screened for enhanced resistance to desiccation as described above to identify clones which stably transmit the mutant phenotype and which therefore carry the appropriate genomic DNA fragment.

Bacteria comprising DNA fragments which are associated with enhanced viability after desiccation are further characterized. In one embodiment, deletion mutagenesis is used to identify the minimal DNA sequences that are responsible for the enhanced viability. These sequences are then subcloned for further functional analysis. The association of a suspected mutation with resistance to desiccation is validated by demonstrating that loss of a positive genomic clone results in the loss of desiccation resistance while retransformation with the positive genomic clone results in gain of desiccation resistance. After a series of subcloning and functional analyses, a DNA fragment can be sequenced to identify genes (e.g., fragments of DNA comprising open reading frames) responsible for the enhanced resistance against desiccation. In addition, mutations responsible for enhanced resistance can be retrieved by random insertion mutagenesis with a selectable marker followed by preparation of a bacteriophage P1 transducing library and subsequently followed by P1 transduction into a nonmutant host.

Generating Competent Cells from Selected Mutants

In one embodiment, the mutant cells which are resistant to desiccation are rendered competent for transformation by exogenous nucleic acids.

Methods of Making Competent Cells

Desiccation resistant mutant cells are first grown in a medium which supports cell proliferation (cell-growth medium), as described above. In one embodiment, the cell-growth medium used is supplemented to comprise additional growth-promoting agents (e.g., vitamins, sugars, ions, and the like). It should be obvious to those of skill in the art that a variety of media can be used, and that such media are encompassed within the scope of the invention.

Incubation temperatures for growing cells can vary from 10° C. to 42° C., but preferably ranges from 20° C. to 40° C. In one embodiment according to the invention, cells are grown with shaking to promote aeration, at 100 to 500 revolutions per minute (rpms). In a preferred embodiment, cells are grown to early to mid log phase, or to early stationary phase, as detected by visual inspection (e.g., for optimal turbidity) or by sampling aliquots of media and determining the optical density (OD) of the media (e.g., at 550 nm, using a spectrophotometer) to select cells having an OD between 0.1 to 2.0. Media can be reinoculated with cells which have reached mid to late stationary phase to reinitiate log phase growth.

In one embodiment, cells at a desired stage of growth are collected (e.g., by centrifugation, filtering, allowing cells to settle, by size exclusion chromatography, and the like) and resuspended, to be washed at least one time, in a suitable transformation buffer, as desribed above. Preferably, the cells are resuspended in transformation buffer which has been pre-cooled to 4° C. and in one embodiment, cells are incubated in transformation buffer for at least 2-60 minutes.

Mutant cells can also be made competent by exposure to electrical pulses which create temporary holes in the cells' plasma membranes (see, e.g., Potter, Anal. Biochem. 174: 361-73, 1988, and U.S. Pat. No. 6,096,549, the entireties of which are incorporated herein by reference). Therefore, in one embodiment, cells are grown in cell-growth medium to mid or late log phase or to early stationary phase and collected (e.g., by centrifugation). Cells are then resuspended and washed at least one time in an electroporation buffer (e.g., 10%-15% glycerol, 90% distilled water, v/v), and placed in a chamber of an electroporation device (e.g., Cell-Porator™, from Life-Technologies; BIO-RAD Gene Pulser®), avoiding air bubbles during the placement process. Cells are exposed to an electrical pulse which varies depending upon the cell type and the size of the container in which the cells are placed. In one embodiment, E. coli cells are rendered permeable by exposure to 1.5 to 2.5 kV (25 uF, 200 ohms) (see, e.g., Dower, et al., Nucleic Acids Res. 16: 6127-6145 (1988), the entirety of which is incorporated herein by reference).

Methods of making competent cells can be selected to suit a user's needs. For example, when transforming cells with supercoiled plasmid DNA, generally any method known in the art will provide an acceptable number of transformants (e.g., 1 per agar plate). However, for clones comprising unstable or less stable sequences (e.g., LTR sequences and inverted repeats), it may be desirable to alter growth conditions to enhance the stability of the cells, i.e., such as by growing cells at lower temperatures (25° C. to 30° C.) in rich medium (e.g., TB broth) and by terminating growth before the cells reach late stationary growth phase. Alternatively, or additionally, mutant cells can be derived from cells whose genotypes minimize rearrangements of unstable sequences (e.g., such as STBL strains). Where limiting amounts of cloned sequences are to be introduced into a cell, transformation buffers can be additionally supplemented by agents for enhancing transformation efficiency, including, but not limited to, hexamine cobalt chloride, sodium succinate, RbCl, and the like (see, as discussed in U.S. Pat. No. 4,981,797, the entirety of which is incorporated by reference herein).

Additional methods of generating competent cells are described in: Kushner, In: Genetic Engineering: Proceedings of the International Symposium on Genetic Engineering, Elsevier, Amsterdam, pp. 17-23 (1978); Norgard, et al., Gene 3: 279-292 (1978); Jessee, et al., U.S. Pat. No. 4,981,797; and at http://www.protocol-online.net/molbio/DNA/transformation.html, the entireties of which are incorporated by reference herein.

Generating Room Temperature Stable Competent Cells

In one embodiment, competent cells prepared by any of the methods described above, or by any methods known in the art, are desiccated prior to contacting with exogenous DNA. For example, in one embodiment, mutant cells are rendered competent, then subjected to desiccation as described above and stored until the cells are rehydrated and exposed to exogenous DNA. In one embodiment, the cells are stored for at least one month.

In one embodiment, the cells are desiccated in the presence of a water soluble glass-forming matrix material as described above.

In one embodiment according to the invention, drying conditions are selected which provide a Tg of greater than or equal to 20° C., greater than or equal to 25° C., greater than or equal to 30° C., greater than or equal to 35° C., and preferably greater than or equal to 45° C. In a more preferred embodiment, conditions are selected which result in a Tg of greater than or equal to 60° C.

In one embodiment, drying is performed using a stepwise temperature increase from 4° C. to 30° C. (i.e., above freezing) over a period of 48 hours. However, room temperature stable competent cells can be generated without temperature steps, and viability is actually enhanced 30% upon drying at a uniform temperature, as measured by plating cells which have been dried using temperature steps, counting the number of colonies formed, and comparing these numbers to the numbers of colonies formed from plated cells which have not been exposed to temperature steps. In one embodiment cells are dried at room temperature. In a preferred embodiment, cells are dried at a temperature within the range of 15° C.-30° C. In a more preferred embodiment, cells are dried at 30° C.

Drying times can be varied to achieve an optimal Tg. In one embodiment, the drying time ranges from 2-48 hours. In a preferred embodiment, the drying time ranges from 6-24 hours. In a more preferred embodiment of the invention, cells are dried at 30° C. from 6-48 hours.

In one embodiment, competent cells are dried under vacuum, to maximize the amount of glass matrix-cell mixture formed in the minimum amount of time, thereby maximizing cell viability. In one embodiment, it is preferred that the glass matrix-forming material-cell mixture be dried at higher than atmospheric pressure. Pressure is optimized to provide the highest Tg and cell viability while providing a product that dries in an intact form (e.g., without forming bubbles). In a preferred embodiment of the invention, the glass-matrix-cell mixture is dried at 1000-4000 mtorr, and preferably at 3000 mtorr. After drying is completed and a satisfactory Tg is obtained, dried cells are stored in sterile containers at room temperature until use. In one embodiment according to the invention, cells are packaged in a form suitable for shipping, for example, by storing the cells in sealed pouches in the presence of desiccant.

Transforming Desiccation-Resistant Competent Cells

Desiccation-resistant mutant cells can be transformed with exogenous DNA after having been previously frozen or desiccated, or, can be made competent immediately prior to transformation (e.g., less than 2 hours before exposure to exogenous nucleic acids). In one embodiment, desiccation-resistant mutant cells which have been exposed to a transformation buffer are collected by centrifugation, and resuspended in a solution comprising glass-forming matrix material. The cells are subsequently dried, for example, by exposure to a vacuum under pressure.

In one embodiment, desiccation-resistant cells which have been desiccated (e.g., for storage) are rehydrated for use in subsequent transformation procedures. In one embodiment, the dried competent cells are resuspended in an appropriate amount of water which does not lyse the cells; i.e., generally, at least a volume of water equal to the volume of stored competent cells. Cells may be further diluted in buffer (e.g., transformation buffer) or cell growth media. In one embodiment, where cells are dried in the presence of a glass-forming matrix, cells are rehydrated, collected (e.g., by centrifugation), and washed at least one time in a transformation medium or cell growth medium, to remove or substantially dilute, residual glass matrix forming material (e.g., to 5% w/v or less).

In one embodiment, rehydrated competent cells according to the invention are used in transformation procedures by contacting the cells with nucleic acids, preferably comprising a selectable marker gene (e.g., a gene encoding resistance to an antibiotic or expressing a detectable polypeptide, or enzyme which can catalyze a detectable reaction, such as β-galactosidase), and plating the cells on a plate which comprises a selection media (e.g., an antibiotic or substrate for the enzyme).

Nucleic acids encompassed within the scope of the invention, include, but are not limited to, nucleic acid sequences that encode functional or non-functional proteins and fragments of those sequences, as well as nucleic acids which comprise non-coding sequences (e.g., regulatory sequences, such as promoters or enhancers). The nucleic acids may be natural (e.g., isolated from cells) or synthetic nucleic acids (e.g., obtained by PCR or mutagenesis of isolated nucleic acids, or chemically synthesized). The nucleic acids can be circular, linear, or supercoiled. Although not limited to particular sizes, in some embodiments, the nucleic acids used to transform the cells according to the invention range from 1.0 kb to 300 kb.

In one embodiment, competent cells which have been contacted with nucleic acids are incubated for 2 minutes to 2 hours at 4° C.-30° C. Contacted cells are plated onto agar plates comprising a suitable selection media, either directly, or after dilution in a cell growth medium (which can also be further incubated to promote cell growth). In one embodiment of the invention, cells are heat shocked at 20-42° C. for 30 seconds to 2 minutes, prior to plating.

In one embodiment, transformation efficiencies of the desiccation-resistant cells generated range from 105 transformants/μg/DNA to 1012 transformants/μg DNA, while the viability of the cells comprises at least 10% of the viability of cells prior to drying. In one embodiment, the viability of the cells comprises at least 20%, at least 30%, at least 50% or 100% of the viability of cells prior to drying.

In a further embodiment of the invention, desiccation-resistant cells which have been rendered electrocompetent and desiccated are rehydrated and exposed to one or more electrical pulses (1.5-2.5 kV) in the presence of nucleic acids. As above, transformed cells can be directly plated or plated after dilution in cell culture media. In one embodiment, the cells are gently resuspended in SOC medium (e.g., 2 ml of 20% glucose and 1 ml of 2M Mg per 100 ml of SOB medium) after electroporation.

In one embodiment, transformation efficiencies of electrocompetent desiccation-resistant cells range from 10⁵ transformants/μg/DNA to 10¹² transformants/μg, while the viability of the cells comprises at least 10% of the viability of cells prior to drying. In another embodiment, the viability of the cells comprises at least 20% or at least 30% of the viability of cells prior to drying. In one embodiment, the transformation efficiency of rehydrated cells which have been exposed to at least one electrical pulse is increased relative to cells which have not been subject to drying and rehydration. In another embodiment, transformation efficiencies of the cells are at least three times as great as the transformation efficiency of cells which have not been subjected to drying and rehydration.

Additional factors can be manipulated to enhance the transformation efficiency of pulsed cells such as the electrical field strength, the pulse decay time, the pulse shape, the temperature at which electroporation is conducted, the type of cell (e.g., SURE™ cells, XL1-Blue MRF′™, and Electro Ten Blue Cells are particularly suited for electroporation), the type of suspension buffer, and the concentration and size of the nucleic acid to be transferred. Optimization parameters are discussed, for example, in Andreason and Evans, Analytical Biochemistry 180: 269-275 (1988); Sambrook, et al., In Molecular Cloning: a Laboratory Manual, 2nd Edition, eds. Sambrook, et al. (Cold Spring Harbor Laboratory Press) pp. 1.75 and 16.54-16.55 (1989); Sambrook, et al. 1987; Stratagene Instruction Manual for Epicurian Coli™ Electroporation-Competent Cells 1997; the entireties of which are incorporated by reference herein). In one embodiment, sugars are added to enhance the electroporation efficiency of the cells (e.g., 0.1% and 5.0% w/v of non-polar aldoses and aldose alcohols).

Producing Recombinant Proteins Using Desiccation-Resistant Competent Cells

In a further embodiment, the invention provides a method of producing recombinant proteins (e.g., proteins expressed by the exogenous nucleic acids which have been used to transform the cells). In this embodiment, competent desiccation-resistant mutant cells which have been transformed with a nucleic acid encoding a protein of interest are grown in a cell-growth medium under conditions in which the cell will express the protein (e.g., the protein may be expressed constitutively by the cell or under inducing conditions, such as during exposure to a selected temperature or a chemical agent, such as IPTG). The protein is then isolated from the cultured cells and purified, e.g., by lysing the cells (e.g., with lysozyme, exposure to a detergent, by sonication, or by some other method), fractionating cellular components, and selecting for fractions of these components which have any of: a desired enzymatic activity, immunological activity, physical characteristic (e.g., molecular mass, spectroscopic properties, and the like), and/or other biological activity.

Fractionating can be performed using affinity column chromatography where an antibody is available for a protein/antigen of interest, by size exclusion chromatography to select proteins within a certain size range, by ammonium sulfate precipitation, polyethylene glycol precipitation, or by using combinations of these methods. Methods of purifying recombinant proteins from bacterial cells are well known in the art (see, e.g., Sambrook, et al., supra, and www.protocolonline.net/molbio/Protein/protein_purification.htm#Protein Extraction).

Packaging and Storage of Dried Desiccation-Resistant Mutant Cells

Dried cells can be packaged and stored in containers at room temperature until use. For specific applications, the cells can be suitably stored in a closed/sealed moisture barrier, or a rigid/sealed container in the presence of desiccant. A variety of desiccants can be used to reduce the water content of the cells, including, but not limited to, calcium sulfate, silica, certain clays, and polyacrylic acid derivatives. For applications which require sterile conditions, cells are stored in sterile pouches in the presence of desiccant.

Desiccation resistant mutant cells stored in this way can typically remain viable for a long period of time at 20° C. or above (e.g., at least one month).

For applications such as for agriculture and aquaculture, the cells can be formulated to form a biomass with plant growth media or feed.

Kits

The invention further provides kits comprising room temperature stable desiccation-resistant competent cells. In one embodiment according to the invention, a kit is provided which comprises room temperature stable desiccation-resistant competent cells in a container for shipping which does not comprise ice or any other frozen packing material. In another embodiment of the invention, room temperature stable competent cells are packaged in a sealed pouch and optionally provided along with a desiccant. In a further embodiment of the invention, cells are provided along with a sample of lyophilized supercoiled plasmid DNA which serve as a control to monitor the transformation efficiency of the competent cells. Additional reagents can also be provided for use in transforming the competent cells, such as a substrate for a marker enzyme which is expressed by a nucleic acid to be transformed (e.g., X-Gal); antibiotics, restriction enzymes to detect signature restriction sites in a cloning vector, and the like.

### EXAMPLES

### Mutagenesis and Selection for Mutants Able To Withstand Desiccation

E. coli strains XL1-Blue MRF′ and ElectroTen Blue were mutagenized as follows.

A 50 ml culture of logarithmically growing cells were collected and washed twice with buffer (50 mM NaCl+10 mM MgCl₂) and resuspended in the same. 10 ml aliquots were transferred to sterile petri dishes and subjected to UV light in a Stratalinker at 100,000 uJ, 125,000 uJ, 150,000 uJ, and 200,000 uJ. The mutagenized cells were then allowed to recover overnight by addition of a cell-growth medium, NZY medium.

The cells were then pooled and competent cells prepared. To generate desiccation-resistant electrocompetent cells, ElectroTen Blue cells were collected, washed twice with 4% trehalose, and resuspended in 1/50 volume of 20% trehalose for drying. Desiccation was performed at 30° C. at 3000 mTorr overnight. For chemically competent cells, XL1-Blue MRF′ cells were cultured and collected, washed once with 4% trehalose, and suspended in FSB (without glycerol). After 20 minutes on ice, the cells were then collected, washed once with 4% trehalose, and collected again. These cells were resuspended in 1/20 volume of 20% trehalose for drying under the same conditions described above.

After drying, the mutagenized cells (“mutant population”) were rehydrated in H₂O and serial dilutions were plated for cells that survived the desiccation process. A plate corresponding to approximately 10⁶ survivors was selected and all of the colonies on the plate were pooled. These pooled cells were grown and prepared for competency and drying as before. Cells were plated and 10⁵ survivors were selected. This process was repeated (each time selecting 10-fold fewer survivors) until 100 colonies were obtained. Each of the 100 were analyzed to determine the viability of their clonal progeny upon desiccation and rehydration, and in some cases for transformation efficiency.

TABLE 1
Isolation of Desiccation Resistant Mutants

| | | Relative Transformation |
|---|---|---|
| Strain | % survival | Efficiency* |
| XL1-Blue (control) | 10 | 0.3 |
| XA2 | 90 | 1.0 |
| XA47 | 20 | 0.4 |
| XA49 | 100 | 6.0 |
| XB40 | 80 | 6.0 |
| XB46 | 60 | 0.5 |
| ElectroTen Blue (control) | 30 | NT** |
| BA4 | 50 | NT |
| BA98 | 90 | NT |
| XL10-GOLD (control) | 8 | NT |
| AT4 | 100 | NT |
| AT7 | 100 | NT |
| AT8 | 100 | NT |
| AT9 | 100 | NT |
| AT37 | 100 | NT |
| AT40 | 100 | NT |
| AT54 | 100 | NT |
| AT66 | 75 | NT |
| AT67 | 80 | NT |
| BT71 | 100 | NT |
| BT72 | 50 | NT |
| BT87 | 100 | NT |

*compared to control strains
**NT: Not tested

XL1-Blue Mutants: Of the 100 screened, 6 demonstrated improved efficiency of survival.

ElectroTen Blue Mutants: Of the 100 screened, 2 demonstrated improved survival

XL10-Gold Mutants: Of the 100 screened, 12 demonstrated improved survival.

Variations, modifications, and other implementations of what is described herein will occur to those of ordinary skill in the art without departing from the spirit and scope of the invention as claimed. Accordingly, the invention is to be defined not by the preceding illustrative description but instead by the spirit and scope of the following claims.

1. A method of isolating a mutant cell that exhibits at least 10% survival after desiccation and rehydration, the method comprising growing cells in a cell-growth medium, desiccating the cells, rehydrating the cells, selecting cells that survive desiccation and re-hydration, and isolating isolation of a mutant cell having resistance to desiccation from said selected cells.

2. The method of claim 1, wherein said selecting comprises at least one step of determining the viability of cells after desiccation.

3. The method of claim 2, wherein said step of determining comprises plating selected cells on a plate comprising cell-growth medium.

4. The method of claim 3, wherein said step of determining further comprises counting the number of colonies formed on said plate.

5. The method of claim 1, wherein the step of isolating comprises cloning said cell.

6. The method of claim 5, wherein said cloning is performed by plating said selected cells, and obtaining a single colony comprising a clone of cells.

7. The method of claim 5, wherein said cloning is performed by limiting dilution of said selected cells.

8. The method of claim 1, wherein cells are mutagenized prior to desiccating.

9. The method of claim 1, wherein said cells are microorganisms.

10. The method of claim 9, wherein said microorganisms are gram-negative bacteria.

11. The method of claim 10, wherein said bacteria are E. coli.

12. The method of claim 1, wherein the steps of desiccation and selecting are performed at least twice.

13. The method of claim 12, wherein the steps of desiccation and selecting are performed at least six times.

14. The method of claim 5, wherein said isolated cell produces progeny cells which exhibit greater than 10% viability after desiccation and rehydration.

15. The method of claim 5, wherein said isolated cell produces progeny cells which exhibit greater than 10% viability after desiccation and rehydration.

16. The method of claim 5, wherein said isolated cell produces progeny cells which exhibit greater than 30% viability after desiccation and rehydration.

17. The method of claim 5, wherein said isolated cell produces progeny cells which exhibit greater than 50% viability after desiccation and rehydration.

18. The method of claim 5, wherein said cloned cell produces progeny cells which exhibit greater than 100% viability after desiccation and rehydration.

19. The method of claim 1, wherein said cells are desiccated in the presence of a glass-forming matrix.

20. A cell produced by the method of claim 1, which exhibits resistance to desiccation.
